(19) United States
(12) Patent Application Publication
(10) Pub. No.: US 2022/0226638 A1
(43) Pub. Date: Jul. 21, 2022

(54) Methods and Devices for Wireless Deep Brain Stimulation

(71) Applicant: Sensoria Therapeutics, Inc., Irvine, CA (US)

(72) Inventor: Ross Tsukashima, Irvine, CA (US)

(73) Assignee: Sensoria Therapeutics, Inc., Irvine, CA (US)

(21) Appl. No.: 17/235,811

(22) Filed: Apr. 20, 2021

Related U.S. Application Data
(60) Provisional application No. 63/018,224, filed on Apr. 30, 2020.

(51) Int. Cl. A61N 1/05; A61N 1/372; A61N 1/36; A61N 1/378; A61N 1/375

(52) U.S. Cl. CPC A61N 1/0534; A61N 1/37235; A61N 1/37514; A61N 1/36128; A61N 1/3787; A61N 1/37229

(57) ABSTRACT

Methods and devices for wireless deep brain stimulation.

This application claims priority to U.S. Provisional Application 63/018,224 filed Apr. 30, 2020.

### FIELD OF THE INVENTIONS

The inventions described below relate to devices and methods that provide treatment for various diseases in the field of wireless deep brain stimulation.

### BACKGROUND OF THE INVENTIONS

Deep brain stimulation (DBS) technology has shown promise for treatment of movement and affective disorders such as Parkinson's disease, epilepsy, essential tremor and dystonia. Deep brain stimulation is accomplished by placing a neurostimulation lead connected to a pulse generator within the brain, near or contacting the brain structures that control motor functions, such as the subthalamic nucleus (STN). Typical treatment protocols use cylindrical probes with electrode assemblies disposed on the distal tip of the probes. The electrode assembles include circumferential electrodes or a number of electrodes arranged around the circumference of cylindrical probe, and provide omnidirectional or limited directional stimulation due to the cylindrical shape of the electrode of brain tissue proximate the tip of a probe. The electrodes on the tip of the probe may be placed in various locations within the brain, and may be operated to stimulate various parts of the brain. Because the circumferential electrodes or electrode arrays of the prior art provide omnidirectional or partial omnidirectional stimulation, they may stimulate structures in the brain to uncertain or undesired effect while stimulating desired structures to achieve a desired effect. For example, common side effects during lateral stimulation in STN-DBS include focal muscle contraction and dysarthria as a result of corticobulbar tract activation. There is a need for an electrode assembly that can stimulate desired areas but also simultaneously avoid stimulation of other non-targeted areas within the brain.

Additionally, cylindrical electrode assemblies are hard to place within the brain. The cylindrical electrode assemblies are prone to rotation or spinning and it can also be difficult to determine which parts of the electrode assemblies are live when placed within the brain. Also, the cylindrical electrodes can migrate back and forth within the brain once implanted instead of remaining securely positioned within the brain. Typically these devices require implantation of a battery pack and control pack within the patient's chest with subcutaneous wires threaded up through the neck to the top of the skull and ultimately to the implanted probes. The wires are a common source of irritation and infection. Also, some leads have multiple wires in the probes, making them bulky, stiff and prone to breakage. In addition, the probes are susceptible to limited battery life and electrical interference. The wireless DBS device described below eliminates wires and simplifies installation, reduces or entirely eliminates battery replacement and is operated and maintained easier than prior art DBS devices. Thus, there is a need for an electrode assembly that allows accurate electrode assembly placement within the brain. Also, there is a need for an electrode assembly that provides for better visualization, works easier under MRI, and is placed and oriented easier than previous electrode assemblies.

### SUMMARY

The devices and methods described below provide for improved deep brain stimulation treatment using an electrode assembly that allows for wireless stimulation of areas of the brain. The wireless DBS device is inserted within the brain while the DBS power transmitter assembly is disposed outside of the patient. The DBS power transmitter is operable outside of the patient's body to deliver power and control signals in order to provide power to the wireless DBS device inside the brain. The wireless DBS device has a housing that contains at least one electrode pair that protrudes from a surface of the housing. The wireless DBS device further includes stimulation electronics, optional sensing electronics, a microprocessor, a power converter and an antenna, all contained within the housing. The antenna is preferably disposed entirely within the housing while the electrode pair preferably protrudes from the housing. The DBS transmitter assembly may contain a power source or battery and an external power-transmitting antenna.

A method of performing deep brain stimulation in a patient's brain is also disclosed. The method is performed by inserting an implantable deep brain-stimulating device through the brain. The device is inserted into the patient brain so that the antenna is disposed entirely within the patient brain and does not extend beyond the brain of the patient. Then, a transceiver located outside of the patient is operated to induce a stimulus on the implantable deep brain-stimulating device to produce a brain stimulating current that stimulates the patient brain. Alternatively, the external transceiver can be used to set stimulation values in the device for parameters such as pulse time, frequency and power levels.

### BRIEF DESCRIPTION OF THE DRAWINGS

FIG. 1 illustrates a wireless deep brain-stimulating device within the brain of a patient.

FIG. 2 illustrates the wireless deep brain-stimulating device.

FIG. 3 illustrates an external transceiver for use with the deep brain-stimulating device.

FIG. 4 is a perspective view of the wireless deep brain-stimulating device.

### DETAILED DESCRIPTION OF THE INVENTIONS

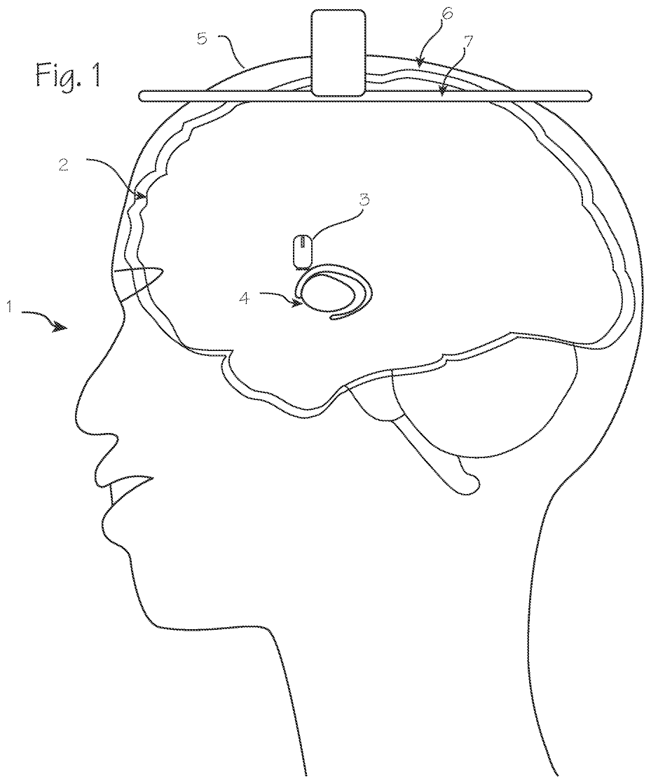

FIG. 1 illustrates a patient 1 with a condition requiring deep brain stimulation (DBS) of the brain 2. FIG. 1 shows the placement of a wireless DBS device 3 within the brain of the patient. The wireless DBS device is disposed within the brain, proximate structures within the brain such as the basal ganglia 4 are shown. The wireless DBS device can be inserted entirely within the brain through the skull 5 and the dura 6. An external DBS power transmitter assembly 7, disposed outside the skull, is operable to power the wireless DBS device. The power transmitter assembly can be coupled with a controller for transmitting control signals to control the wireless DBS device within the brain, and may be coupled with a receiver for receiving data from optional sensors disposed on or within the wireless DBS device 3.

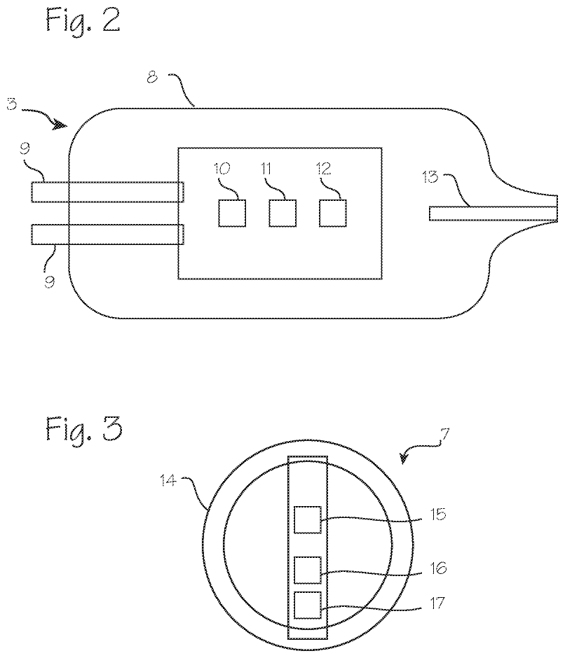

FIG. 2 illustrates the wireless DBS device 3 in detail. The device includes a housing 8 that contains at least one electrode pair 9 that protrudes from a surface of the housing, an optional stimulation or sensing electronics 10, microprocessor 11, and a power converter 12, which includes a RF signal generator and optionally a signal booster, and an antenna 13. The power converter is controlled by the microprocessor. Optionally, an additional antenna could be provided where the first antenna is operable for power reception and the second separate antenna is operable for data transmission and reception. The power converter, antenna, the optional stimulation electronics and microprocessor, are all preferably enclosed within the housing 8. The antenna is preferably disposed entirely within the housing, and thus disposed entirely within the brain when implanted such that the antenna does not come into contact with tissue other than brain tissue, such as the dura or the skull. Alternatively, the antenna may extend beyond the boundaries of the housing, if configured such that, when implanted, the antenna extends into brain tissue but does not extend beyond the brain of the patient and does not extend through either the dura or the skull. The power converter includes a RF signal generator that generates a signal that is to be transmitted to the power transmitting antenna. The transmitter assembly is operable to transmit radiofrequency energy to the deep brain stimulation device to cause the deep brain stimulation device to deliver electrical stimulation through the electrode pair to the brain of the patient. The power converter in turn is operable to convert radiofrequency energy from the external transmitter to power the microprocessor and stimulation electronics. The DBS device does not include a battery and receives power entirely from the external power transmitter and therefore does not require any power leads on the DBS device. The electrode pair protrudes from the housing and is exposed to the brain tissue. The antenna can be a straight wire, a helical coil or a flat coil. The antenna component can be provided in the form of an antenna assembly, with three components (straight wires, helical coils or flat coils) oriented in 3 axes orthogonal to each other to improve reception in any orientation. The DBS device is operable to receive power and, optionally, control signals, from the external power transmitter 7, in order to trigger stimulation of the brain. The optional stimulation electronics 10 can include any printed circuit boards, application specific integrated circuits or other electronic components. The DBS electrodes are resistant to corrosion in the cerebral spinal fluid within and surrounding the brain and can be made of platinum, palladium, silver, titanium and iridium alloys and silver oxide. The materials may also be conductive and non-metallic such as graphite, graphene, pyrolytic or glassy carbon. The DBS electrode can be two-dimensional such as a contact pad or can be three-dimensional such a probe.

FIG. 3 illustrates a top view of an external DBS transmitter assembly 7. The DBS transmitter assembly includes an external power transmitting antenna 14 which may be configured as a loop antenna that is sized for placement around the skull of the patient. The transmitter assembly 7 may contain a power source or battery 15 and, optionally, transmitter electronics 16, which are operable outside of the patient's body to deliver power and control signals to the power converter 12 and microprocessor 11 in order to provide power to the wireless DBS device 3 inside the brain. The transmitter electronics may be part of a transceiver electronics, operable to both transmit control signals to the DBS device and receive signals from the DBS device, where the DBS device includes sensors and electronics for transmitting sensor signals to the transceiver electronics. A control system 17 may be provided to control operation of the transmitter electronics. The DBS transmitter (or transceiver) is activated and operated outside of the patient's brain and employs a remote wireless telecommunication scheme. The transmitter assembly 7 (or the transceiver electronics) is operable to generate and send a signal to the DBS device via the internal antenna 13 to the power converter 12 and microprocessor 11, which in turn are operable to operate the stimulation electronics 10. The stimulation electronics then produce a brain-stimulating electrical stimulus that is delivered through the electrodes to stimulate the patient's brain. The stimulus could be a voltage, current or light that is applied through the stimulation electronics.

The transceiver assembly 7 is located external to the patient, providing the advantage that no surgery is required to replace the transceiver battery. Instead, the external transceiver can be recharged and it can be replaced like a hat, allowing the patient to swap a depleted or broken transceiver with a charged unit. In addition, the external transceiver does not need to be implanted within the patient and provides the additional benefit of no risk of infection or complications based on implantation of the transceiver.

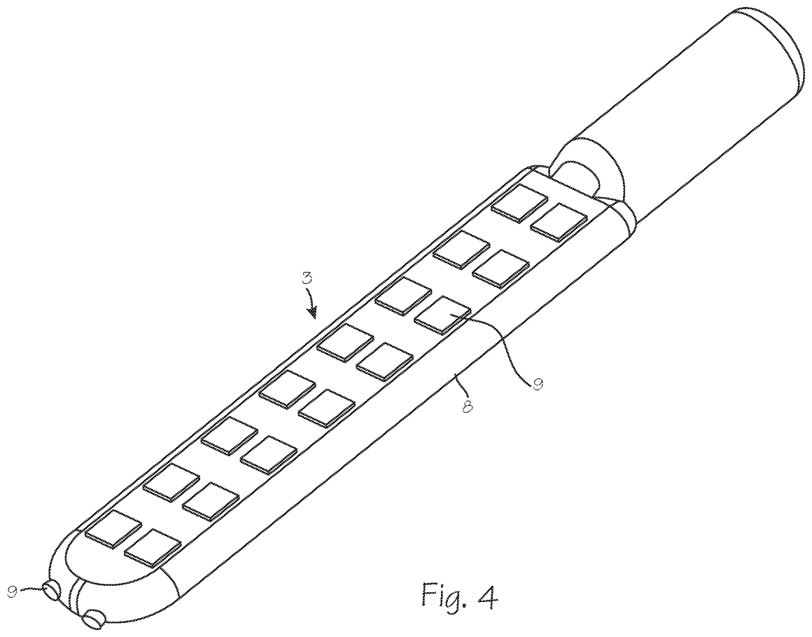

FIG. 4 is a perspective view of the wireless deep brain-stimulating device. The deep brain stimulation device includes a housing 8 having a first end and a second end. The housing includes at least one electrode pair 9 that protrudes from a surface of the housing whereby current can be applied through the electrode pair of the deep brain stimulation device to tissue proximate the electrode(s). The electrode pair can be positioned at the end of the housing or a top or bottom surface. The electrodes may be positioned only on either a top surface of a bottom surface of the housing with an opposite side not containing any electrodes to provider isolation to preferred parts of the brain. The housing has an asymmetrical cross sectional shape in a transverse axis of the deep brain stimulation device to prevent rotation of the deep brain stimulation device after placement. The deep brain stimulation device may be paddle-shaped in order to prevent the migration or rotation of the deep brain stimulation device when installed in the brain. Alternatively, the deep brain stimulation device may be triangular, rectangular or other non-symmetrical shape. The beneficial aspects of this form of the probe of the deep brain-stimulating system may be used in combination with the wireless features described above, and may also be used with wired deep brain-stimulating system to achieve the anti-migration effect of the configuration.

In use, a surgeon will implant the DBS device within the brain of a patient so that the electrode pair contacts the patient brain, in a region subject to stimulation to affect symptoms of a disease such as Parkinson's disease.

The surgeon will implant the device with the antenna 13 disposed entirely within the brain. Preferably, the device will be provided in a form in which the antenna 13 is disposed entirely within the housing of the DBS device. The surgeon will implant the device, in either case, such that the antenna 13 remains entirely with brain tissue, and does to penetrate the dura or the skull of the patient. After the DBS device is implanted, surgeon, the patient, or a later care-giver, will operate the external DBS transmitter assembly, disposed proximate the external surface of the skull as necessary, to provide power to the DBS device and transmit control signals to the DBS device, as desired to affect symptoms of a disease subject to treatment by the DBS device. The patient may wear a transceiver that is configured in the form of a loop antenna that may be shaped as a piece of wearable headwear (a hat or headband). The transceiver is programmed and operated outside of the patient's body to cause the DBS device to deliver a prescribed dosage of electrical impulses to treat a variety of conditions and diseases. Alternatively, the external transceiver can be used to set stimulation values in the device for parameters such as pulse time, frequency and power levels. When stimulation is desired, the transceiver electronics generate and send a signal via the internal antenna through the microprocessor and to the stimulation electronics within the housing to produce a brain-stimulation to the patient brain.

Two or more DBS devices may be implanted in the same patient, and operated independently by the control system and transmitter electronic/transceiver electronic 16 by applying unique address numbers to each of the plurality of DBS devices, and operating the control system and transmitter electronics to broadcast unique address(es) of individual and broadcast control signals along with power signals, to cause an individual DBS device to generate stimulation pulses and deliver stimulation pulses to the brain through the electrode pair.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A system for wireless deep brain stimulation of a brain of a patient, said system comprising:
a deep brain stimulation device comprising:
a housing having a first end and a second end the housing having an electrode pair configured to contact brain tissue when implanted in the brain of the patient;
an antenna configured to be disposed entirely within the brain of the patient when implanted in the brain of the patient, such that the antenna does not extend beyond the brain of the patient and does not penetrate the dura of the patent; and
an external transmitter assembly comprising an antenna operable to transmit radiofrequency energy to the deep brain stimulation device to cause the deep brain stimulation device to deliver electrical stimulation through the electrode pair to the brain of the patient.

2. The system of claim 1, wherein the deep brain stimulation device further comprises:
stimulation electronics operable to produce electrical stimulation for transmission to the brain of the patient through the electrode pair;
a microprocessor for controlling the stimulation electronics to cause the stimulation electronics to produce said electrical stimulation;
a power converter operable to convert radiofrequency energy from the external transmitter to power the microprocessor and stimulation electronics.

3. The system of claim 2 wherein the power converter further comprises an RF signal generator operable to generate a signal and transmit the signal from the antenna of the power converter to the external power transmitting antenna.

4. The system of claim 3 wherein the power converter further comprises a signal booster operable to amplify the signal generated by the RF signal generator and transmit the signal from the antenna of the power converter to the external power transmitting antenna.

5. The system of claim 1 wherein the housing does not including a battery within the housing.

6. A method of performing deep brain stimulation on a patient's brain, said method comprising:
providing an implantable deep brain stimulating device comprising a housing having a first end and a second end the housing having an electrode pair that protrudes from a surface of the housing, stimulation electronics enclosed with the housing, a microprocessor enclosed within the housing, a power converter enclosed within the housing and an antenna in contact with the patient brain;
inserting the implantable deep brain stimulating device through the brain and into the patient brain so that the antenna is disposed entirely within the patient brain and does not extend beyond the brain of the patient;
wirelessly operating a transceiver located outside of the patient to induce a stimulus on the implantable deep brain stimulating device to produce a brain-stimulating current that stimulates the patient brain.

7. The method of claim 6 further comprising the steps of:
inserting a second implantable deep brain stimulation device through the brain and into the patient brain so that the antenna is disposed entirely within the patient brain and does not extend beyond the patient of the brain; and
wirelessly operating the transceiver located outside of the patient brain to induce a voltage on the implantable deep brain stimulating device to produce a brain stimulating current that stimulates the brain independently from the first implantable deep brain stimulating device.

8. A deep brain stimulation device comprising:
an asymmetrical cross sectional housing having a first end and a second end, wherein the asymmetrical cross sectional shape is in a transverse axis of the housing;
an electrode pair on a surface of the housing, the electrode pair configured to contact brain tissue when implanted in the brain of the patient;
an antenna configured to be disposed entirely within the brain of the patient when implanted in the brain of the patient, such that the antenna does not extend beyond the brain of the patient and does not penetrate the dura of the patent; and
an external transmitter assembly comprising an antenna operable to transmit radiofrequency energy to the deep brain stimulation device to cause the deep brain stimulation device to deliver electrical stimulation through the electrode pair to the brain of the patient.

9. The deep brain stimulation of claim 8 further including:
stimulation electronics operable to produce electrical stimulation for transmission to the brain of the patient through the electrode pair;
a microprocessor for controlling the stimulation electronics to cause the stimulation electronics to produce said electrical stimulation;
a power converter operable to convert radiofrequency energy from the external transmitter to power the microprocessor and stimulation electronics.

10. The deep brain stimulation of claim 8 where the deep brain stimulation device is paddle-shaped.
